(19) United States
(12) Patent Application Publication
(10) Pub. No.: US 2010/0304427 A1
(43) Pub. Date: Dec. 2, 2010

(54) SUBSTRATES FOR ADHERING, CULTURING AND ASSAYING CELLS

(76) Inventors: Ronald A. Faris, Elmira, NY (US); Terry T. Goodrich, Corning, NY (US); John S. Peanasky, Big Flats, NY (US); Wanda J. Walczak, Big Flats, NY (US)

Correspondence Address: CORNING INCORPORATED, SP-TI-3-1, CORNING, NY, 14831, US

(21) Appl. No.: 12/788,033

(22) Filed: May 26, 2010

Related U.S. Application Data
(60) Provisional application No. 61/182,369, filed on May 29, 2009.

(51) Int. Cl. C12Q 1/02; C12M 1/34; C12M 1/22; C08J 3/28

(52) U.S. Cl. 435/29; 435/288.3; 435/305.1; 522/153; 522/164

(57) ABSTRACT

The present invention provides substrates useful for culturing cells under low serum or serum free conditions. The substrates are useful for conducting cell-based assays where there is interference from serum proteins. Methods are also provided for using the substrates of the present invention as well as methods for making the substrates of the present invention.

### CLAIMING BENEFIT OF PRIOR FILED U.S. APPLICATION

This application claims the benefit of U.S. Provisional Application Ser. No. 61/182,369, filed on May 29, 2009. The content of this document and the entire disclosure of publications, patents, and patent documents mentioned herein are incorporated by reference.

### BACKGROUND

The present invention relates generally to supports for adhering, culturing and assaying cells in the presence or absence of serum, and particularly to supports having a propylamine derivatized ethylene maleic anhydride layer.

Culturing of adherent mammalian cells often requires the presence of serum and/or extracellular proteins to allow attachment of the cells to a support. However, the presence of these proteins can interfere with cell-based assays and introduce undefined factors into cell culture. Additionally, serum proteins can vary from lot to lot, adding variability to cell-based assays and cell culture.

Another drawback to cell-based assays is seen with respect to the support or matrix that is often required for culturing cells. These supports are often biological materials and therefore are expensive to produce and may be irreproducible to a certain degree. For example, Matrigel™ (BD, Franklin Lakes, N.J.) and collagen sandwich cultures have been used for hepatocyte culture as they maintain superior in vivo like function for extended periods of time. Matrigel™ is derived from the EHS mouse tumor, and may exhibit lot to lot variability, although it supports cell function. The utility of this substrate for human liver ADME/Tox applications may be limiting since (a) the source is from a different species, and hence may not provide a good model for human responses; and, (b) is variable because it is not synthetically derived. Likewise, neither substance can be easily and reproducibly processed into a 3D scaffold which has good stability and mechanical properties.

There is a need for a substrate having a defined composition while still maintaining cell function for an extended period of time, allowing for more predictive cell based assays. A need for a synthetically derived, reproducible substrate for ADME/Tox and other applications also exists.

### SUMMARY

In one aspect of the present invention there is provided a substrate for cell culture and cell-based assays comprising a support, a tie layer comprising an aminoalkylsilane or derivatives thereof, for example aminoalkylsilsesquioxane or aminopropylsilane, attached to the support, a synthetic polymer layer attached to the tie layer, the synthetic polymer layer comprising a plurality of ionizable hydrophilic groups, ionizable hydrophobic groups, or combinations of these, and wherein at least 50% of the ionizable hydrophilic and ionizable hydrophobic groups are inactivated or blocked.

In another aspect of the present invention there is provided a synthetic substrate for adhering and culturing cells comprising a support, a tie layer, and a derivatized aminopropyl ethylene maleic anhydride coating on at least one surface of the support and wherein the derivatized aminopropyl ethylene maleic anhydride coating has a surface contact angle of from about 10° to about 80°, or from about 10° to about 30°.

In a further aspect of the present invention there is provided a method for performing cell culture and cell-based assays comprising adhering at least one cell to a substrate in the absence of serum proteins, wherein the substrate comprises a support, a tie layer comprising an aminoalkylsilane or derivatives thereof for example aminoalkylsilsesquioxane or aminopropylsilane, attached to the support, a synthetic polymer layer attached to the tie layer, the synthetic polymer surface comprising a plurality of ionizable hydrophilic groups, ionizable hydrophobic groups or combinations of these, and wherein the coatings and support are optionally irradiated, culturing the cell on the substrate without serum proteins and performing a cell-based assay.

In yet another aspect of the present invention, there is provided a method of producing a substrate for cell culture and cell-based assays comprising attaching a tie layer to a support, wherein the tie layer comprises an aminoalkylsilane, an aminoalkylsilsesquioxane or derivatives thereof, attaching a synthetic polymer layer to the tie layer, the synthetic polymer surface comprising a plurality of ionizable hydrophilic groups, ionizable hydrophobic groups or combinations thereof. In embodiments, the surface may be irradiated.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description present embodiments of the invention, and are intended to provide an overview or framework for understanding the nature and character of the invention as it is claimed. The accompanying drawings are included to provide a further understanding of the invention, and are incorporated into and constitute a part of this specification. The drawings illustrate various embodiments of the invention, and together with the description serve to explain the principles and operations of the invention.

### BRIEF DESCRIPTION OF THE DRAWINGS

FIG. 1 is a schematic representation of a general structure of the surface of the substrate, according to embodiments of the present invention;

FIG. 2 is a schematic representation of a substrate having an amino propylsilane tie layer derivatized with ethylene maleic acid, according to embodiments of the invention;

FIG. 3 is a schematic representation of the synthesis of a substrate, according to embodiments of the present invention;

FIG. 4 is a schematic representation of the synthesis of a substrate, according to embodiments of the present invention;

FIG. 5 is a schematic representation of the synthesis of a substrate, according to embodiments of the present invention;

FIG. 6 shows a series of IR spectra comparing the effect of irradiation and/or hydrolysis on the surface of the substrate, according to embodiments of the present invention;

FIG. 7 shows a series of IR spectra showing the effect of the hydrolysis on the surface of the substrate in the presence of different buffers, according to embodiments of the present invention;

FIG. 8 shows a series of IR spectra showing the effect of different irradiation treatments on the surface of the substrate, according to embodiments of the present invention;

FIG. 9 is a bar graph showing attachment of HepG2 C3a liver cells on different substrates according to embodiments of the present invention;

FIG. 10 is a bar graph showing attachment of HepG2 C3a liver cells on different substrates at day 1 and retention of HepG2 C3a liver cells on different substrates at day 7, according to embodiments of the present invention;

FIG. 11 is a bar graph showing the gene expression profile of primary liver cells cultured with serum on different substrates according to embodiments of the present invention;

FIG. 12 is a bar graph showing the gene expression profile of primary liver cells cultured without serum on different substrates according to embodiments of the present invention;

FIG. 13 shows a series of IR spectra showing the effect of gamma irradiation after hydrolysis with phosphate buffered saline on the surface of the substrate, according to the present invention;

FIG. 14 comprises a series of IR spectra showing the effect of gamma irradiation after hydrolysis with borate buffer on the surface of the substrate, according to embodiments of the present invention;

FIG. 15 is a bar graph showing the effect of different derivatization conditions of APS/dEMA with n-propylamine on cell culture under serum free conditions in embodiments of the present invention;

FIG. 16 is a bar graph showing the effect of different derivatization conditions of APS/dEMA with n-propylamine on cell culture under serum full conditions in embodiments of the present invention;

FIG. 17 is a bar graph showing the effect of various cell culture media on cell cultures using embodiments of the substrate of present invention;

FIG. 18 is a bar graph showing the effect of different levels of derivitization with n-propylamine and treatment with low gamma irradiation of APS/dEMA on cell culture under serum and serum free and low gamma conditions in embodiments of the present invention; and

FIG. 19 is a bar graph showing the effect of different levels of derivitization with n-propylamine and treatment with high gamma irradiation of APS/dEMA on cell culture under serum and serum free and high gamma conditions in embodiments of the present invention.

### DETAILED DESCRIPTION

In embodiments, the present invention provides a substrate for cell culture and cell-based assays comprising a support where the support comprises a tie layer attached to the support and a synthetic polymer layer attached to the tie layer. The tie layer may be an aminoalkylsilane such as aminoalkylsilsequioxane or aminopropylsilane or derivatives thereof while the synthetic polymer may comprise a plurality of ionizable hydrophilic groups, ionizable hydrophobic groups, or combinations thereof. The coatings may be irradiated by UV treatment or by gamma radiation as non-limiting examples. As a non-limiting example, the substrate may comprise a support with a derivatized aminopropylsilane ethylene maleic anhydride surface coating. The present invention also provides methods for making the substrate as well as for using the substrate for cell culture and/or cell-based assays.

In embodiments of the present invention, the substrate allows for adherence and culturing of mammalian cells in the absence of serum or extracellular proteins. The lack of serum and extracellular proteins results in less interference and background in cell-based assays using the substrate of the present invention. Moreover, in embodiments, the substrate of the present invention is a synthetic substrate which is economical to produce with a high degree of reproducibility. In contrast, substrates for culturing mammalian cells which are obtained from naturally occurring materials, including tissue and cell lines, may be costly and suffer from lot to lot variability.

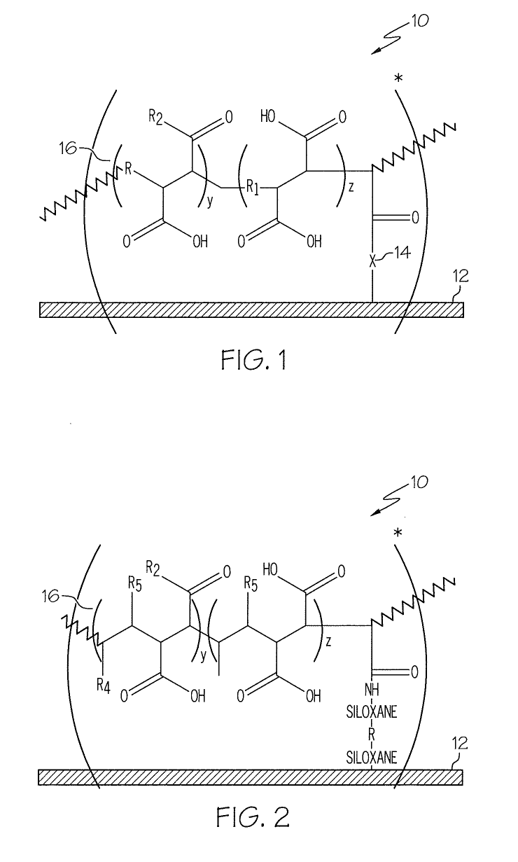

As shown in FIG. 1, the substrate 10 of the present invention may comprise a support 12, a tie layer 14 attached to the support 12 and a synthetic polymer layer 16 attached to the tie layer 14. The synthetic polymer layer 16 may comprise R and R₁ wherein R and R₁ represent independent monomers in the polymer layer 16. It will be appreciated that R and R₁ are shown for illustrative purposes and that the polymer layer 16 may comprise any number of independent polymers; i.e. R, R₁, R₂ . . . Rₙ. Alternatively, the polymer layer 16 may comprise a homopolymer where R and R₁ may be the same. The number of monomers in the polymer layer, illustrated by y and z in FIG. 1, may vary wherein y and z are integers. There may be a string of the same monomer, wherein x or y is greater than 1, or there may be individual monomer units that alternate along the polymer layer 16. The synthetic polymer layer 16 may further comprise R₂ wherein R₂ may be a hydrophilic or hydrophobic group, which may be ionizable, of the polymer layer 16. Ionizable hydrophilic groups are groups that form ions or carry a positive or negative charge under conditions of use. Ions are generated during the inactivation treatment step, such as hydrolysis and/or irradiation treatment. Ionizable hydrophilic groups were relatively hydrophilic prior to treatment. Ionizable hydrophobic groups were relatively hydrophobic prior to treatment. The synthetic polymer layer 16 may comprise a plurality of ionizable hydrophilic groups and hydrophobic groups besides the single R₂ group shown in FIG. 1 for simplicity. Examples of ionizable hydrophilic groups include but are not limited to carboxyl, amino, and aldehyde. Examples of hydrophobic groups include but are not limited to methyl, ethyl, propyl, butyl, (or higher order nmer) and phenyl which may or may not have pendant groups with double or triple bonds. An example of an ionizable hydrophobic group would include n-propyl amine. The hydrophilic and hydrophobic groups may be involved in adhering the cells to the substrate in the absence of serum and/or extracellular proteins. Moreover, R₂ may be unreactive toward cells or biological molecules and not form covalent bonds with such. Cells or other biological molecules may be exposed to the polymer layer 16 wherein the cells attach to the substrate 10 through non-covalent interactions such as, but not limited to ionic, hydrophobic or Van der Waals interactions.

The substrate 10 may be treated by hydrolysis and/or irradiation. Both treatments, alone or in combination may increase the hydrophilicity of the surface of substrate 10 by, for example, increasing the number of carboxylate anions and other charged moieties at the synthetic polymer layer 16 on the surface of the substrate 10 as compared to untreated substrate 10. For example, the substrate 10 of FIG. 1 is irradiated as indicated by “*”. The hydrolysis or irradiation treatment may increase the number of carboxyl moieties by hydrolyzing any anhydrides or other reactive groups. The substrate 10 of the present invention adheres cells through non-covalent interactions. Surprisingly, unlike substrates and surfaces of the prior art, cells are able to function and grow on the substrate 10 in the presence of low levels of serum or in the absence of serum.

Other substrates in the prior art are similar to the present invention in that they comprise a support, a tie layer and a synthetic polymer layer. In particular U.S. Patent Application Publication Nos: 2006/0257919 and 2007/0154348 disclose substrates having a support, a tie layer and a synthetic polymer layer comprising a plurality of ionizable groups. The substrates of the prior art are meant to be used to bind biomolecules such as protein and nucleic acids and therefore require that a majority of moieties of the synthetic polymer layer be reactive. By “reactive” it is meant that the moieties of the synthetic polymer layer are able to covalently bind another molecule without the introduction of catalysts, enzymes or any other condition that would drive the covalent binding to another molecule. For example, an anhydride moiety is a reactive moiety because it will react with, for example, primary amine moieties without any additional reaction drivers. The greater the number of biomolecules that can be attached to the substrate, the better the performance of the substrate. In this regard, the prior art teaches that only 10% to 50% of the reactive groups may be inactivated or blocked before binding of the biomolecules. “Inactivated” or “blocked” groups are those that cannot covalently bind another molecule without the introduction of catalysts, enzymes or any other condition that would drive the formation of a covalent bond. In contrast, the present invention relies on non-covalent interactions such as ionic bonding, hydrogen bonding and Van der Waal interactions to adhere cells to the substrate. In embodiments, the synthetic polymer layer of the substrates of the present invention may be derivatized to convert reactive groups to inactivated groups. Moreover, the gamma radiation and hydrolysis treatments of the present invention may convert reactive groups (such as anhydrides) to inactivated charged groups. For example, maleic anhydride is converted to maleic acid in the substrate of the present invention by gamma radiation and/or hydrolysis. The synthetic polymer layer of the substrate of the present invention has greater than about 50% of the reactive moieties either inactivated or blocked. Therefore, because of the high percentage of inactivated or blocked groups, the substrates of the present invention defeat the purpose of the substrates of the prior art.

An exemplary embodiment of the substrate 10 of the present invention is shown in FIG. 2 where the tie layer 14 on the support 12 is aminopropylsilsesquioxane. The synthetic polymer layer 16 further comprises R₄ and R₅ which may each independently be a hydrophobic or hydrophilic group such as, but not limited to H or styrene. The hydrophilic and hydrophobic groups of R₄ and R₅ may be involved in adhering the cells to the substrate in the absence of serum and/or extracellular proteins. Moreover, R₄ and R₅ may not covalently bind cells or biological molecules to the substrate.

The support 12 may include, but is not limited to, a cell culture surface, a cell-based assay surface, a cell culture vessel, a multiwell plate, a microplate, a slide, a strip well, a Petri dish, a flask, a multi-layer cell culture device (such as Hyperflask® or Cellstack®, both available from Corning Incorporated, Corning, N.Y.), a cell chamber in a fluidic device or any other material that is capable of attaching to the tie layer 14. The support 12 may be flat, fibrous, or 3-dimensional in nature. In one aspect, when the support 12 is a microplate, the number of wells and well volume will vary depending upon the scale and scope of the analysis. Other examples of supports 12 for use in the substrate 10 of the present invention may include, but are not limited to, a cell culture surface such as a 384-well microplate, a 96-well microplate, 24-well dish, 6-well dish, 10 cm dish, or a T75 flask.

For optical or electrical detection applications, the support 12 may be transparent, impermeable, or reflecting, as well as electrically conducting, semiconducting, or insulating. For biological applications, the support material may be either porous or nonporous and may be selected from either organic or inorganic materials.

In a further aspect, the support 12 may comprise a plastic, a polymeric or co-polymeric substance, a ceramic, a glass, a metal, a crystalline material, a noble or semi-noble metal, a metallic or non-metallic oxide, an inorganic oxide, an inorganic nitride, a transition metal, or any combination thereof. Additionally, the support 12 may be configured so that it can be placed in any detection device. In one aspect, sensors may be integrated into the bottom/underside of the support 12 and used for subsequent detection. These sensors could include, but are not limited to, optical gratings, prisms, electrodes, and quartz crystal microbalances. Detection methods may include fluorescence, phosphorescence, chemiluminescence, refractive index, mass, and electrochemical. In one aspect, the support is a resonant waveguide grating sensor.

In a further aspect, the support 12 may be composed of an inorganic material. Examples of inorganic support materials may include, but are not limited to, metals, glass or ceramic materials. Examples of metals that can be used as support materials may include, but are not limited to, gold, platinum, nickel, palladium, aluminum, chromium, steel, and gallium arsenide. Glass and ceramic materials used for the support material may include, but are not limited to, quartz, glass, porcelain, alkaline earth aluminoborosilicate glass and other mixed oxides. Further examples of inorganic support materials may include graphite, zinc selenide, mica, silica, lithium niobate, and inorganic single crystal materials.

In a further aspect, the support 12 may be composed of an organic material. Organic materials useful herein may be made from polymeric materials due to their dimensional stability and resistance to solvents. Examples of organic support materials may include, but are not limited to, polyesters, such as polyethylene terephthalate and polybutylene terephthalate; polypropylene, polyvinylchloride; polyvinylidene fluoride; polytetrafluoroethylene; polycarbonate; polyamide; poly(meth)acrylate; polystyrene, polyethylene; cyclic polyolefins ; ethylene/vinyl acetate or copolymers, or other known organic support materials.

In one aspect of the present invention, a tie layer 14 (see FIG. 1) may be attached to the support 12. The tie layer 14 may comprise an amine such as, but not limited to, aminosilane. In a further aspect, the tie layer 14 may be derived from a straight or branched-chain aminosilane, aminoalkoxysilane, aminoalkylsilane, aminoarylsilane, aminoaryloxysilane, or a derivative or salt thereof. In a further aspect, the tie layer may be derived from 3-aminopropyl trimethoxysilane, N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoxane.

In another aspect of the present invention, a synthetic polymer layer 16 may be attached, either covalently or electrostatically, to the tie layer. The synthetic polymer layer 16 may have a plurality of ionizable hydrophilic and/or ionizable hydrophobic groups (i.e. R₂ in FIG. 1 or R₂, R₄ and R₅ in FIG. 2). In one aspect of the present invention, the ionizable group may be converted to a negatively charged group such as, but not limited to, a carboxylate or an ion pair or positively charged groups such as but not limited to amino groups. Non-limiting examples of ionizable groups may be maleic acid, acrylic acid or methacrylic acid. The hydrophobic groups may have alkyl, cycloalkyl, aryl and allyl groups such as, but not limited to, propyl, ethyl or phenyl. The ionizable hydrophilic and hydrophobic groups may be involved in adhering the cells to the substrate 10 in the absence of serum and/or extracellular proteins. Moreover, the ionizable hydrophilic and ionizable hydrophobic groups themselves may be inactive in that they may not covalently bind cells or biological molecules to the substrate. Alternatively, the ionizable hydrophilic and ionizable hydrophobic groups may be converted to moieties that may be inactive in that they may not covalently bind cells or biological molecules to the substrate 10.

In a further aspect of the present invention, less than about 50% of the ionizable hydrophilic and hydrophobic groups of the synthetic polymer layer 16 are reactive groups. Reactive groups are groups that are able to form a covalent bond with another molecule (i.e. biomolecule) without the aid of a catalyst or another compound. In another aspect of the present invention the ionizable hydrophilic and ionizable hydrophobic groups may be treated to provide a substrate having less than 50% reactive groups. Stated another way, in embodiments the ionizable hydrophilic and ionizable hydrophobic groups may be treated to provide a substrate having more than 50% of the ionizable hydrophilic and ionizable hydrophobic groups inactivated. Treatment of the ionizable hydrophilic and hydrophobic groups by hydrolysis or irradiation may inactivate the reactive groups wherein they can no longer form a covalent bond with another molecule. Alternatively, the ionizable hydrophilic and ionizable hydrophobic groups may be derivatized or blocked such that they are no longer reactive. In an exemplary embodiment, the synthetic polymer layer 16 may have from about 10% to about 40% reactive groups. Or, stated another way, in embodiments, the synthetic polymer layer 16 may have from about 90% to about 60% inactivated groups, or greater than 50% inactivated groups.

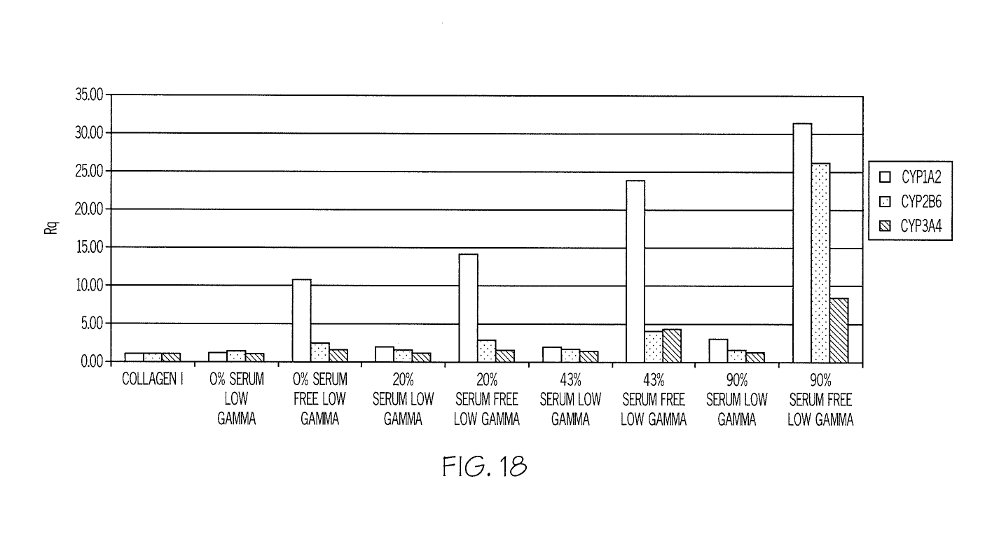

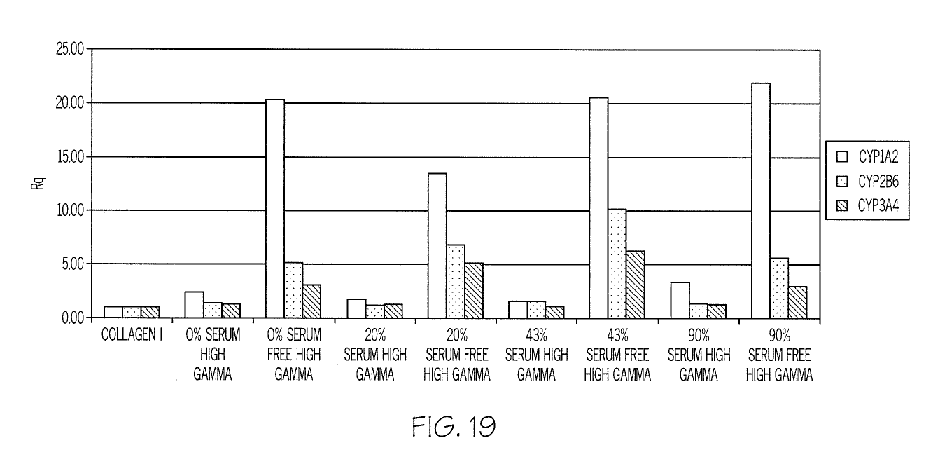

It has been found that better cell function under serum free conditions is obtained the lower the number of reactive groups on the synthetic polymer layer 16 of the substrate 10. This is illustrated FIGS. 18 and 19. FIGS. 18 and 19 show the basal response of CYP1A2, CYP2B6 and CYP3A4 levels in cells cultured under serum free conditions on substrates having varying degrees of derivitization. CYP1A2, CYP2B6 and CYP3A4 are the cytochrome P450 enyzmes 1A2, 2B6 and 3A4. The substrates were also irradiated under low (FIG. 18) or high (FIG. 19) gamma radiation. At 90% derivatization, cells responded well to being cultured under serum free conditions after low gamma treatment (see FIG. 18). Those of ordinary skill in the art will recognize that, while these conditions were optimal for these cells in these conditions, other cells, or other functions of these same cells, map perform optimally in different conditions.

In another aspect of the invention, the synthetic polymer layer 16 may have a copolymer comprising first monomer having an ionizable group and a second monomer having a hydrophobic group. For example, the synthetic polymer layer 16 may be, but may not be limited to, a copolymer of maleic acid and alkyl monomer such as ethylene. The ionizable group may have maleic acid, acrylic acid, methacrylic acid or combinations thereof. In a further aspect, the synthetic polymer layer 16 may have a terpolymer of a first monomer that has an ionizable group, a second monomer and a third monomer that carries a hydrophobic pendant group. The second and third monomers may comprise ethylene, styrene, octadecene, methyl vinyl ether, ethylene, isobutylene or combinations thereof. In embodiments, the second and third monomers may be a vinylester, vinylamide, acrylamide, acrylate groups or combinations thereof and may have a hydrophobic pendant group where the hydrophobic pendant group may be alkyl (including cycloalkyl), aryl, or allyl groups or combinations thereof. A non-limiting example may be a terpolymer of maleic acid, alkyl monomer such as ethylene, and propylacrylamide group.

Alternatively, the synthetic polymer layer 16 may comprise a terpolymer or tripolymer comprising a first monomer that has an ionizable group, a second monomer having a hydrophobic group and a reactive third monomer that can allow for further modification of the surface with small molecules such as peptides and biological ligands. The reactive third monomer may be ethylene. The reactive third monomer may be present in concentrations such that the synthetic polymer layer, and thus the substrate, has less than 50% reactive groups or moieties.

In yet another aspect of the present invention, the synthetic polymer layer 16 may comprise, but is not limited to, poly(ethylene-alt-maleic anhydride, poly(methyl vinyl ether-alt-maleic acid), poly(styrene-alt-maleic acid), maleic acid vinyl acetate copolymer, the anhydride derivatives thereof or any mixture thereof. The anhydride derivatives may be ionized and/or hydrolyzed to provide negatively charged carboxylate moieties in the synthetic polymer layer 16. In an exemplary aspect of the present invention the synthetic polymer layer is derivatized with an amino moiety such as, but not limited to, n-propyl amine. The amount of derivatization may be from about 20 mol % to about 50 mol % or from about 20 mol % to about 90 mol %. In additional embodiments, the amount of derivatization is greater than 20 mol %, greater than 40 mol %, greater than 45 mol %, greater than 50 mol %, greater than 60 mol %, greater than 70 mol %, or greater than 80 mol %.

In another aspect of the present invention, the surface contact angle of the synthetic polymer layer 16 may be important for the serum-free or low-serum attachment and growth of cultured cells. The surface contact angle of the synthetic polymer layer 16 may be from about 10° to about 80°, about 10° to about 70°, about 10° to about 60°, about 10° to about 40°, or about 10° to about 30°. The surface contact angle reflects the hydrophobicity of the synthetic polymer layer 16. Generally, it is understood in the art that the higher the contact angle, the more hydrophobic the surface, while the lower the contact angle the more hydrophilic the surface.

The substrate 10 of the present invention may further comprise small molecules that promote cell adhesion to the substrate or other desirable cell characteristics. The small molecules may be attached to the synthetic polymer layer either covalently or electrostatically. Non-limiting examples of small molecules include, but are not limited to, peptides, proteins, biological ligands, sugars such as glucose, galactose, N-acetylgalactose, aminated versions of galactose and N-acetyl galactose, polysaccharides and combinations thereof. Examples of peptides that aid in cell adhesion may be YIGSR, RGD, polylleucine (i.e. leucine dimers or trimers) or mimics thereof. The small molecules may specifically interact with hepatocyte receptors such as the ASGPR or the EGF receptor or mimics thereof. For example, galactose and N-acetylgalactose interact with the ASGPR while hydrophobic peptides or moieties, such as oligomers of leucine interact with the EGF receptor. It will be appreciated however, that a substrate in and of itself without the additions of the small molecules, provides adequate cell attachment and function.

The small molecules may be attached to the synthetic polymer on the substrate either directly or indirectly through the use of a spacer. Such spacers are known in the art and may be chosen based on length and/or functional groups for binding the small molecules to the substrate. Non-limiting examples of such spacers may be alkyl or PEG spacers (C3-C18).

Embodiments of the present invention also provide methods for making the disclosed substrate. In embodiments, the methods comprise the steps of attaching a tie layer to a substrate where the tie layer may comprise an amine such as, but not limited to, aminosilane. In a further aspect, the tie layer may be derived from a straight or branched-chain aminosilane, aminoalkoxysilane, aminoalkylsilane, aminoarylsilane, aminoaryloxysilane, or a derivative or salt thereof. In a further aspect, the tie layer may be derived from 3-aminopropyl trimethoxysilane, N-(beta-aminoethyl)-3-aminopropyl trimethoxysilane, N-(beta-aminoethyl)-3-aminopropyl triethoxysilane, N′-(beta-aminoethyl)-3-aminopropyl methoxysilane, or aminopropylsilsesquixoxane.

The tie layer may be attached to the support either covalently or electrostatically. The tie layer may be disposed on the substrate using various techniques known in the art. In an embodiment, the tie layer may be dispensed, dip coated, sprayed from solution, vapor deposited, spin coated, screen printed, or robotically pin printed or stamped on the substrate. This may be done either on a frilly assembled support or on a bottom insert (e.g., prior to attachment of the bottom insert to a holey plate to form a microplate). Alternatively, there may be a second tie layer wherein the first tie layer attaches to the support through the second tie layer.

The optional second tie layer may be attached to the first tie layer using various adherence techniques known in the art. In an embodiment, the second tie layer may be dispensed, dip coated, sprayed from solution, vapor deposited, spin coated, screen printed, or robotically pin printed or stamped on the substrate on the first tie layer. This may be done either on a fully assembled support or on a bottom insert (e.g., prior to attachment of the bottom insert to a holey plate to form a microplate).

The method further comprises the step of attaching a synthetic polymer to the tie layer, either covalently or electrostatically, to form a synthetic polymer layer. In an embodiment, the synthetic polymer layer may be dispensed, dip coated, sprayed from solution, vapor deposited, spin coated, screen printed, or robotically pin printed or stamped on the tie layer. This may be done either on a fully assembled support or on a bottom insert (e.g., prior to attachment of the bottom insert to a holey plate to form a microplate). The synthetic polymer layer may comprise a plurality of ionizable hydrophilic and hydrophobic groups. In one aspect of the present invention, the ionizable group may be converted to a negatively charged group such as, but not limited to, a carboxylate or an ion pair. Non-limiting examples of ionizable groups may be oxalic acid, maleic acid, succinic acid, carboxylic acid, glutaric acid, adipic acid, pimelic acid, acrylic acid or methacrylic acid. Alternatively, the ionizable group can be converted to a positively charged group such as but not limited to an amino group. The hydrophobic groups may comprise alkyl, cycloalkyl, aryl and allyl groups such as, but not limited to, propyl, ethyl or phenyl. Moreover, the ionizable hydrophilic and hydrophobic groups themselves may be inactive in that they may not covalently bind cells or biological molecules to the substrate. Alternatively, the ionizable hydrophilic and hydrophobic groups may be converted to moieties that may be inactive in that they may not covalently bind cells or biological molecules to the substrate. In other words, the ionizable hydrophilic and ionizable hydrophobic groups may be inactivated. For example, an anhydride group may be inactivated through covalent bonding to n-propyl amine. This covalent bonding is considered “derivatization.” The resulting structure is ionizable, hydrophobic and inactivated.

The method may further comprise the step of ionizing or converting any reactive groups such as anhydrides to hydrophobic or charged groups of the synthetic polymer layer. Alternatively, the synthetic polymer layer may be treated to increase the hydrophilicity as reflected by decreasing the surface contact angle. This may allow for non-covalent adherence of the cells and biological moieties. In one exemplary embodiment, the method may further comprise the step of hydrolyzing the substrate surface either prior to or after irradiation. The hydrolysis of the substrate surface may further enhance the cell binding properties of the substrate. After attachment of the tie layer and the synthetic polymer layer, the substrate is allowed to incubate in the buffer for at least 5 minutes or for as long as 90 minutes. The buffer may include, but is not limited to phosphate buffered saline (PBS), other phosphate buffers and borate buffers. The buffers may have a concentration of at least about 100 mM and the pH may be from about 7.0 to about 10.0. Following hydrolysis the buffer may be removed by washing with deionized water or it may be removed without washing.

In an alternate exemplary embodiment, the surface groups of the synthetic polymer layer may be ionized or converted to the desired moieties by low and high gamma sterilization. The high gamma sterilization may be from about 25 to about 40 kGY. The low gamma sterilization may be from 10-18 kGY. While not wishing to be bound by theory, hydrolysis, irradiation or both, may change the surface contact angle of the substrate and consequently, the physical properties of the substrate. The irradiated substrate may have a surface contact angle of the synthetic polymer layer ranging from about 10° to about 80° or from about 10° to about 30°.

In a further aspect, the method may also comprise the step of attaching to the substrate small molecules prior to ionizing or hydrolyzing the hydrophobic or hydrophilic groups of the synthetic polymer layer via irradiation or buffer treatment. Small molecules are contemplated as those that promote cell adhesion to the substrate or other desirable cell characteristics. The small molecules may be attached to the synthetic polymer layer covalently or electrostatically prior to the irradiation of the substrate. An aqueous solution comprising the small molecule or a mixture thereof is added to the substrate. The small molecule-containing solution may be added to the wells if the substrate is a multiwell plate or other container or the substrate may be submerged in the solution. For consistent attachment on the substrate surface, the substrate and solution may be shaken gently or moved in any way to keep the solution moving across the substrate. The attachment step may be done at any temperature that results in attachment and does not degrade the small molecules or the underlying substrate. The substrate surface may then be dried by methods known in the art such as, but not limited to, air drying or drying under low heat that will not degrade the small biological molecules. It will be known to those skilled in the art which conditions to use for attaching the desired small molecule(s).

In a further aspect the concentration of the small molecule solution may be from about 0.001 to about 0.005 mM for peptides and proteins and from about 5 mM to about 15 mM for sugars. It will be appreciated that the attachment of small molecules to surfaces is well known in the art and therefore the desired small molecule may be attached to the substrate of the present invention without undue experimentation. Non-limiting examples of small molecules include, but are not limited to, peptides, proteins, biological ligands, sugars such as glucose, galactose, N-acetylgalactose, aminated versions of galactose and N-acetyl galactose, polysaccharides and combinations thereof. Examples of peptides that aid in cell adhesion may be YIGSR, RGD, peptides containing an RGD sequence, polylleucine (i.e. leucine dimers or trimers) or mimics thereof. The small molecules may specifically interact with hepatocyte receptors such as the ASGPR or the EGF receptor or mimics thereof. For example, galactose and N-acetylgalactose interact with the ASGPR while hydrophobic peptides or moieties, such as oligomers of leucine interact with the EGF receptor. It will be appreciated however, that the substrate in and of itself without the additions of the small molecules, provides adequate cell attachment and function.

The small molecules may be attached to the synthetic polymer on the substrate either directly or indirectly through the use of a spacer. Such spacers are known in the art and may be chosen based on length and/or functional groups for binding the small molecules to the substrate. Non-limiting examples of such spacers may be alkyl or PEG spacers (C3-C18).

The method further comprises the step of sterilizing the substrate before using the substrate in application requiring sterile conditions, such as culturing cells. The sterilization step may be desired when using the substrate for cell culture. The substrate may be irradiated by UV treatment or by gamma radiation. The UV treatment may be at 365 nm for 30 minutes to 2 hours. Alternatively, the UV treatment may be from 45 minutes to 75 minutes. In another aspect, the substrate may be irradiated by from about 5 to about 60 kGY or from about 10 to 40 kGY of gamma radiation. The gamma irradiation may either be low gamma sterilization, from about 10 to about 20 kGY or high gamma sterilization, from about 25 to about 40 kGY. It will be appreciated that the low gamma and high gamma irradiation used to ionize the synthetic polymer layer in the previous step may also sterilize the substrate at the same time.

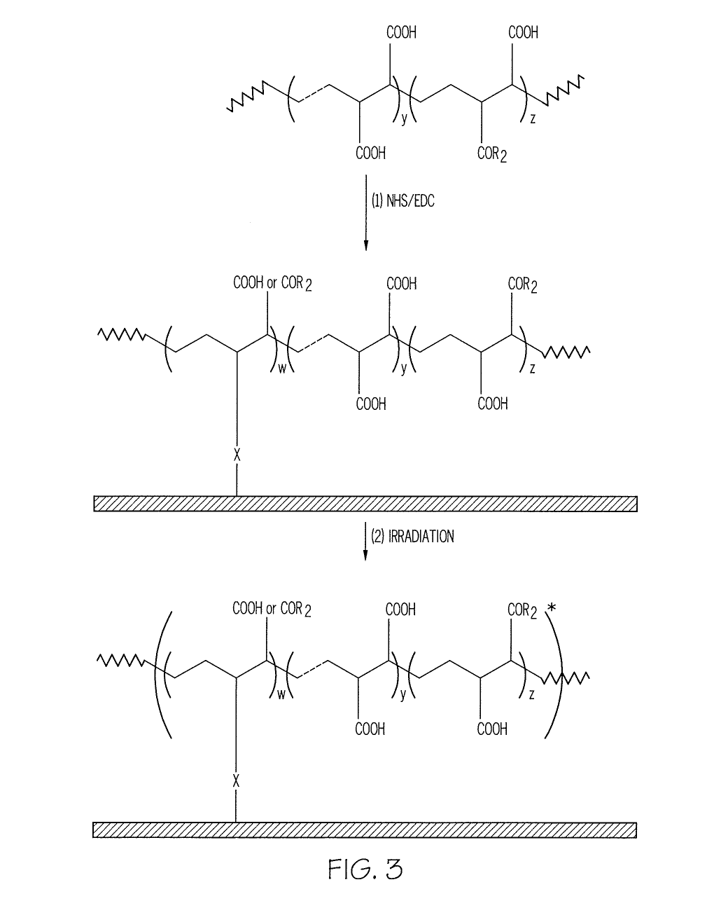

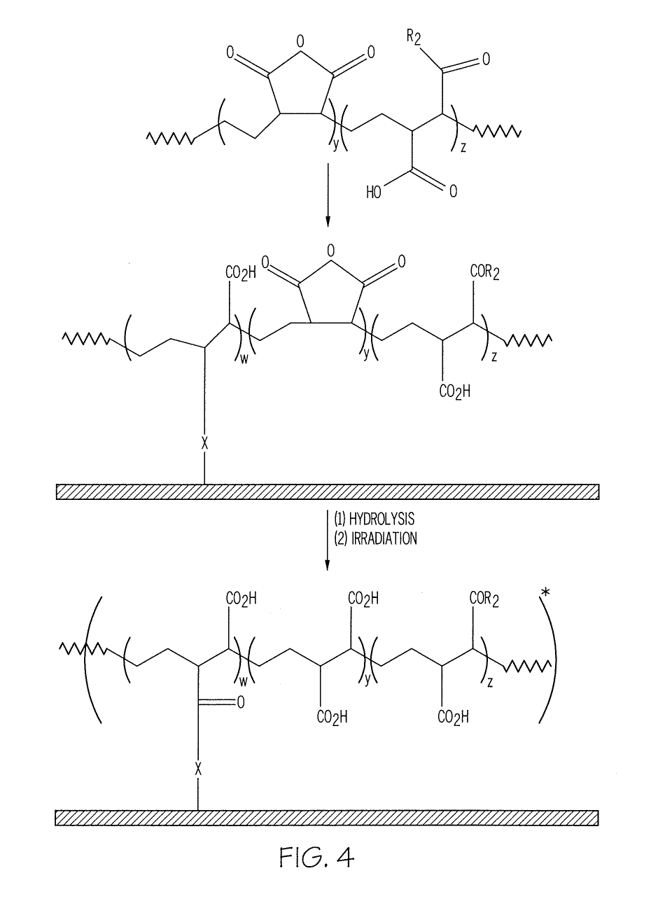

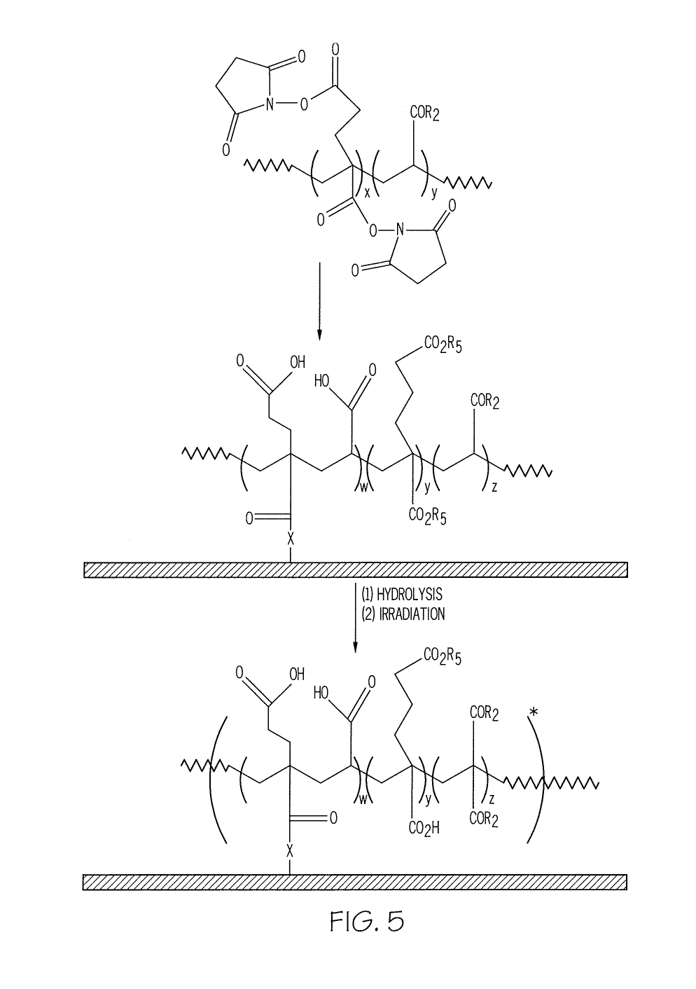

Examples of the method of making the substrate of the present invention are shown in FIGS. 3, 4 and 5. R₂ and R₅ in FIGS. 3, 4 and 5 are as described for FIGS. 1 and 2 above where the star indicates irradiation and w, y and z are integers from about 0 to about 30. It has been found that irradiating the synthetic polymer layer of the substrate changes the properties of the substrate. It is difficult to chemically define the changes that occur during irradiation with any bonds that are typical or characteristic during normal synthesis and which are the same with UV and gamma irradiation per se. Changes in the contact angle of the substrate surface and a conversion of reactive groups to inactive groups are two changes which have been observed. Upon gamma irradiation more carbonyl groups are observed, but not so for UV irradiation. Similarly, gamma irradiation produces a more hydrophilic coating, while UV irradiation produces a more hydrophobic coating. Hence a general (*) was used to describe irradiation. FIG. 3 shows an example of a synthetic route for adhering a synthetic polymer layer comprising hydrolyzed derivatized polymaleic acid to a tie layer using N-hydroxysulfosuccinimide/1-ethyl-3-(3-dimethylaminopropyl) carbodiimide hydrochloride (NHS/EDC) chemistry. In this example, a tie layer is disposed on a substrate. The tie layer may be disposed on the substrate using various techniques known in the art. For example, the support may be dipped in a solution of the tie layer. In a further aspect, the tie layer may be dispensed, sprayed from solution, vapor deposited, screen printed, or robotically pin printed or stamped on the tie layer. This may be done either on a fully assembled support or on a bottom insert (e.g., prior to attachment of the bottom insert to a holey plate to form a microplate). The synthetic polymer layer may be disposed on the tie layer. The synthetic polymer layer may be attached to the tie layer using various techniques known in the art. In embodiments, the synthetic polymer may be dispensed, dip coated, sprayed from solution, vapor deposited, spin coated, screen printed, or robotically pin printed or stamped on the tie layer. This may be done either on a fully assembled support or on a bottom insert (e.g., prior to attachment of the bottom insert to a holey plate to form a microplate). As illustrated in FIGS. 3 and 5, anhydride groups present in the synthetic polymer may be hydrolyzed before being attached to the tie layer. Alternatively, as illustrated in FIG. 4, anhydride groups in the sythetic polymer may be hydrolyzed after the synthetic polymer layer is attached to the tie layer. NHS/EDC is useful for crosslinking carboxylic acid groups with primary amine containing molecules. If the tie layer, X, is a compound such as aminopropylsilsesquioxane containing primary amines, and the polymer contains carboxylic acid groups, NHS/EDC may be utilized to facilitate the attachment of the polymer to the tie layer. NHS/EDC chemistry is known in the art. After attachment of the synthetic polymer layer to the tie layer, the substrate is irradiated to sterilize the substrate.

FIGS. 4 and 5 also include the step of hydrolyzing the substrate before irradiation. FIG. 4 shows the application of a derivatized polymaleic anhydride acid to a tie layer, X, followed by hydrolysis of the unreacted anhydride groups via buffer and subsequent irradiation. FIG. 5 shows the hydrolysis of an N-succinimide ester upon binding to the tie layer, forming polymaleic acid, which is further hydrolyzed upon buffer exposure in a subsequent step.

In one aspect of the present invention there is provided a method for performing cell culture and cell-based assays comprising the steps of adhering at least one cell to embodiments of the substrate of the present invention in the absence of serum or exogenously added proteins and culturing the cells on the substrate in the absence of serum or exogenously added proteins. The cell or cells may be cultured for the amount of time required to reach the desired confluence. This may be from about 1 day to about 14 days. After the cells have reached the desired confluence, the cells may be used in cell based assays directly on the substrate. While cells may be attached to the substrate in the absence of serum or serum proteins, low levels of serum or extracellular proteins may be used. In normal applications, 10% serum in medium is the standard for good cell adherence to a support. In embodiments, “low serum” conditions may be media with less than 10% serum, less than 7% serum, less than 5% serum, less than 2% serum, 0.5% to 2% serum, or no serum. The cells are cultured under standard conditions for the desired cell type. For example, mammalian cells may be cultured at about 37° C. in a CO₂ controlled environment.

The cells may be any cells desired by the skilled artisan. In an embodiment of the invention, the cells are mammalian cells. Examples include primary mammalian hepatocytes, including human, mouse, rat, porcine, rabbit and ovine, as well as cell lines including human cell lines HepG2, HepG2/C3A or FaN2-4.

Any cell-based assay may be used with the substrate of the present invention. It will be appreciated that the choice of a support for the substrate will depend on the type of assay that is desired. For example, a 6-well, or a 96 well plate may be preferred, depending upon the type of assay desired. Additionally, the cell-based assay may be used to determine any number of parameters with regard to the cells. The assay may be to determine the response of a cell or a targeted pathway in a cell to a compound, such as in a drug screen. It may be an assay for toxicity, metabolism or bioavailability for the compounds or molecules of interest. It will be appreciated that the present invention is not limited by an assay method.

### Examples

Advantages and improvements of the present invention will be further demonstrated and clarified by way of the following examples. These examples are illustrative only and are not intended to limit or preclude other embodiments of the present invention.

### Example 1

Synthetic scheme for APS/dEMA substrates: The APS/dEMA substrates were made by applying aminopropylsilane (APS) (Gelest WSA-9911) as a 2% aqueous solution onto glass using a dipcoating or dispensing process. The concentration of the aminopropylsilane was 2%, diluted from its emulsion listed at 20-25% concentration in water. After allowing the aminopropylsilane layer to dry, a derivatized EMA (dEMA) was applied via dipcoating or dispensing. Derivatization of the EMA (polyethylene maleic anhydride (CAS #9006-26-2, Part #188050, Sigma Aldrich Mw=100,000-500,000) took place in an inert atmosphere by addition of a 10 mg/ml propylamine solution in NMP to the EMA solution (14 mg/ml). The glass insert was adhered to a 96 well holey plate using a pressure sensitive adhesive (Arocure 09106).

For those substrates comprising additional small molecules, an appropriate solution was made of peptide, sugar or peptide+sugar mix in phosphate or borate buffer. Typically, a concentration of 10 mM sugar and 0.002 mM concentration of peptide were used. The small molecules used included galactosamine, N-acetylgalactosamine, RGD, YIGSR, Leucine trimer and Leucine dimer. 100 μl of the small molecule solution was placed onto the APS/dEMA substrates on a 96 well microplate using a microchannel pipette and allowed to incubate at RT on a plate shaker until the reaction was complete, typically 60 minutes to overnight. The wells were then rinsed with sterile PBS multiple times. Finally, the plates were irradiated with UV (365 nm light) for 1 hour. It should be noted that hydrolysis of the APS/dEMA synthetic polymer coated surface occurred during the incubation with the small molecule in the buffer solution.

### Example 2

Analysis of the substrate surface. The effect of the irradiation treatment as well as hydrolysis by different buffers on the substrate surface of APS/dEMA was analyzed. The hydrophobic and hydrophilic properties were analyzed by measuring contact angles. Generally, it is understood in the art that the higher the contact angle, the more hydrophobic the surface, while the lower the contact angle the more hydrophilic the surface. A summary of the contact angle measurements obtained is given in Table 1.

TABLE 1
Contact angle measurements on unhydrolyzed
APS/dEMA and various treatments thereof.

| Sample | Contact Angle (degrees) |
|---|---|
| Unhydrolyzed APS/dEMA | 66-76 |
| UV Sterilization (365 nm, 1 hr) | 74-78 |
| Low Gamma Sterilization (10-18 kGy) | 48.3 +/− 3.9 |
| High Gamma Sterilization (25-40 kGY) | 22.02 +/− 1.3 |
| Unhydrolyzed APS/dEMA | 73.2 +/− 2.07 |
| Hydrolyzed with PBS 90 minutes | 13.2 +/− 1.3 |
| (—Ca2+, —Mg2+) pH 7.4 | |
| UV sterilization (365 nm, 1 hr) post PBS | 19.5 +/− 2.13 |
| Unhydrolyzed APS/dEMA | 72.77 +/− 2.91 |
| Hydrolyzed with Borate Buffer 90 | 14.6 +/− 1.3 |
| minutes (100 mM, pH = 9.4) | |
| UV sterilization (365 nm, 1 hr) post | 17.09 +/− 1.6 |
| Borate | |
| Unhydrolyzed APS/dEMA | 73.62 +/− 2.97 |
| Hydrolyzed with Borate Buffer 90 | 63.1 +/− 1.7 |
| minutes (100 mM, pH = 9.4) | |
| Followed with a DI water wash | |
| (overnight) | |
| UV sterilization (365 nm, 1 hr) post | 61.8 +/− 2.9 |
| borate/water | |

The first section of Table 1 compares an unhydrolyzed polyethyelene co-alt maleic anhydride that had been derivatized approximately 40% with n-propyl amine to samples that had been either UV sterilized, gamma sterilized at low levels (10-18 kGY) or gamma sterilized at high levels (25-40 kGY). The second section of Table I compares an unhydrolyzed polyethylene co-alt maleic anhydride that had been derivatized approximately 40% with n-propyl amine to samples that had either been hydrolyzed with phosphate buffered saline or hydrolyzed with phosphate buffered saline followed by UV sterilization. The third section of the table compares an unhydrolyzed polyethylene co-alt maleic anhydride that had been derivatized approximately 40% with n-propyl amine with samples hydrolyzed with borate buffer or hydrolyzed with borate buffer followed by UV sterilization. Finally, the fourth section of the table compares an unhydrolyzed polyethylene co-alt maleic anhydride that had been derivatized approximately 40% with n-propyl amine to samples hydrolyzed with a borate buffer followed by a deionized water wash or a hydrolysis step followed by UV sterilization.

For the case of UV sterilization of hydrolyzed or unhydrolyzed polyethylene co-alt maleic anhydride that had been derivatized approximately 40% with n-propyl amine for 1 hr at 365 nm, the contact angle increased slightly for both the unhydrolyzed and hydrolyzed surfaces, except when a water wash is used. With gamma irradiation, the contact angle decreased dramatically for the unhydrolyzed surface. There was approximately a two-fold decrease with exposure to low gamma irradiation and approximately a four-fold decrease with exposure to high gamma irradiation. The largest decrease in surface contact angle was observed for samples that were hydrolyzed with PBS or borate buffer without a subsequent water wash. Behavior of surfaces that have been gamma irradiated are similar to the ones that have been UV sterilized post hydrolysis. For example, PBS hydrolysis followed by UV sterilization showed a contact angle of about 10 degrees.

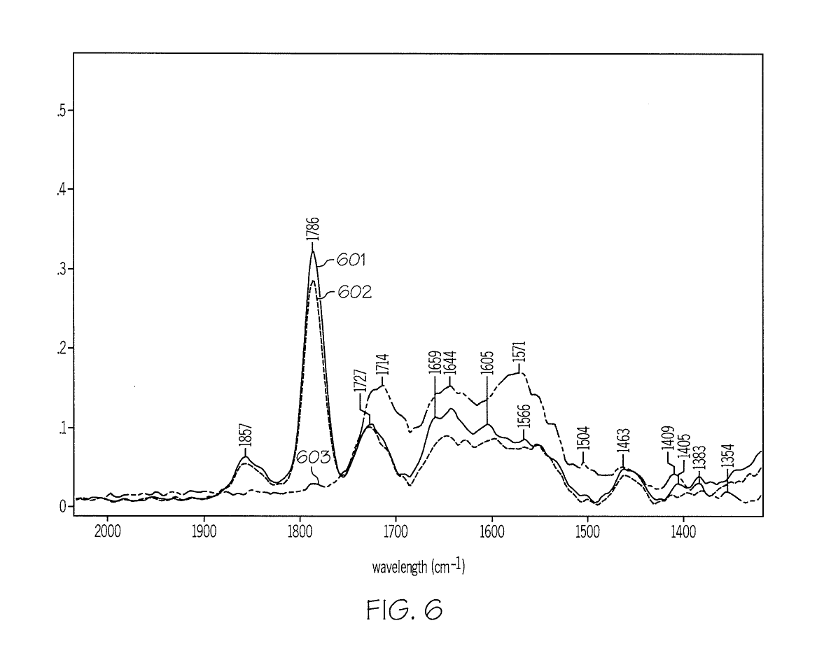

A chemical comparison of various treatments of the substrate surfaces was made by IR analysis of the substrates. FIG. 6 shows infrared spectra of a polyethyelene alt maleic anhydride that had been derivatized approximately 40% with n-propyl amine (APS/dEMA) 601, the same exposed to UV for 1 hr at 365 nm 602 and exposed to phosphate buffered saline to hydrolyze any anhydride groups to acid groups followed by UV exposure for 1 hr at 365 nm 603. The clear absence of peaks at 1786 cm⁻¹ and 1857 cm⁻¹ in the sample that was exposed to phosphate buffered saline followed by UV exposure. 603 was due to the hydrolysis and resulting loss of the anhydride group. In other words, the anhydride group was inactivated by the treatment. As shown in FIG. 6, UV sterilization alone 602 does not hydrolyze the surface or change the anhydride groups to carboxylic acid groups. Instead it seems to make the surface slightly more hydrophobic as demonstrated by the change in the contact angle (Table 1).

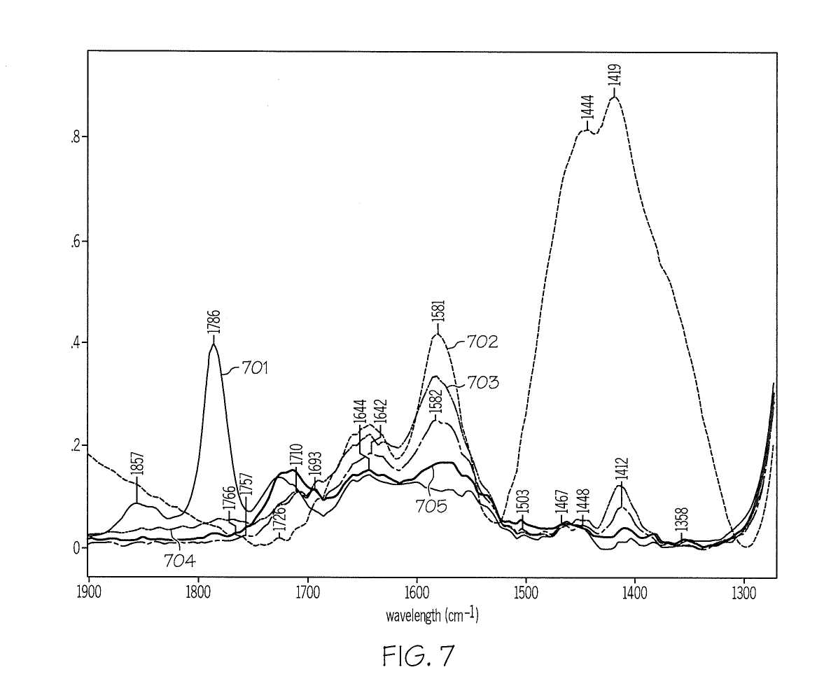

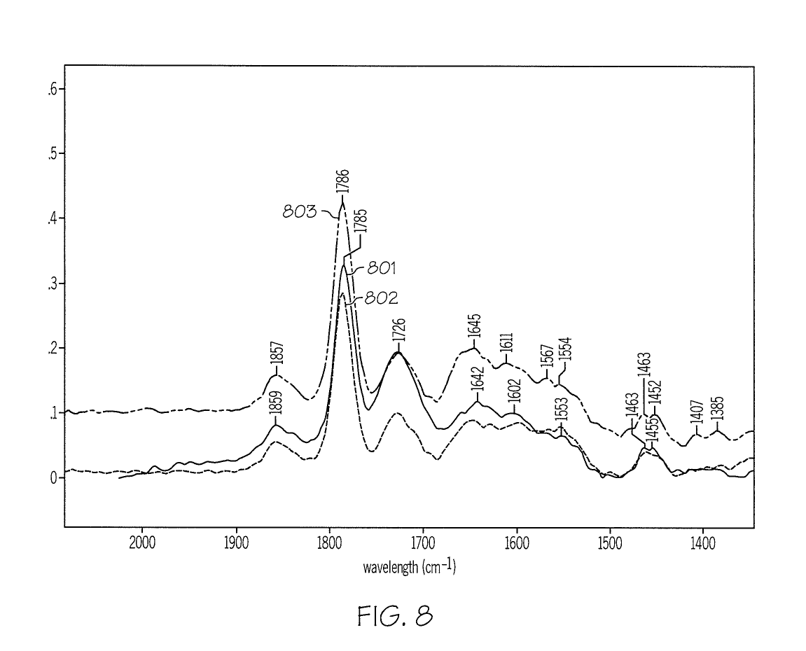

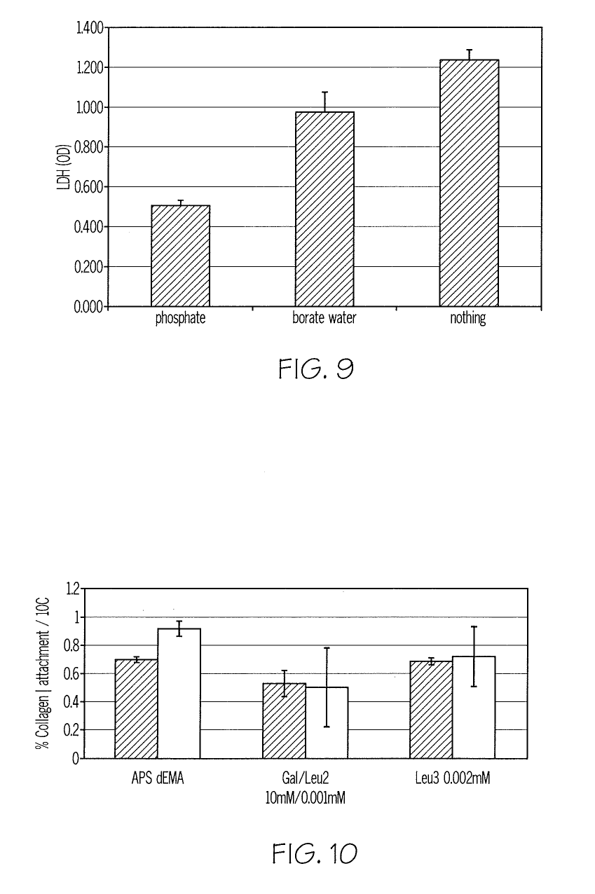

FIG. 7 shows a comparison of unhydrolyzed APS/dEMA with APS/dEMA that has been hydrolyzed with borate buffer, phosphate buffered saline, or borate/water. A UV sterilized APS/dEMA surface that has been hydrolyzed with phosphate buffered saline is also included for comparison. FIG. 7 shows infrared spectra of polyethylene co alt maleic anhydride that has been derivatized approximately 40% with n-propyl amine and untreated 701, hydrolyzed with 100 mM borate buffer (pH=9) 702, hydrolyzed with phosphate buffered saline 703, hydrolyzed with 100 mM borate buffer followed by a deionized water wash 704 or hydrolyzed with phosphate buffer saline followed by a deionized water wash and sterilization with UV for 1 hr at 365 nm 705. The different buffer treatments indicate complete hydrolysis of the anhydride peaks at 1857 and 1786 cm⁻¹ as well as changes in the strength of the carboxylate anion at 1581 cm⁻¹. Large changes in the bands at 1444 and 1419 cm⁻¹ upon borate buffer exposure which are eliminated with a water wash are unexplained at this time. Differences in the 1589, 1444 and 1410 cm⁻¹ bands are seen for the various hydrolysis methods, indicating different surfaces are exposed to cells. The 1589 cm⁻¹ band is most likely due to a carboxylic acid salt and indicates that the presence of this group for the different surfaces is highest for borate>phosphate>borate/water>unhydrolyzed. The amount of this group on the surface correlates with cell attachment, as shown in FIG. 9. The absence of the 1786 cm⁻¹ band during hydrolysis indicates the loss of anhydride functionality in the unhydrolyzed surface upon hydrolysis. The peaks at 1444 cm⁻¹ and 1419 cm⁻¹ are most likely due to a complex between boron and nitrogen that is washed away with water.

FIG. 8 shows a comparison of UV sterilized APS dEMA to APS dEMA sterilized at high levels and untreated APS dEMA. FIG. 8 shows infrared spectra of polyethylene co alt maleic anhydride derivatized approximately 40% with n-propyl amine and sterilized with high gamma sterilization 801, UV sterilization for 1 hr at 365 nm 802 or unsterilized 803. Gamma sterilization at 25-40 kGY indicates changes in the 1726 cm⁻¹ carbonyl band relative to the anhydride bands at 1856 and 1786 cm⁻¹ as compared to the unsterilized or UV sterilized cases. Differences in the 1726 to 1780 cm⁻¹ ratio are clearly seen between gamma and UV sterilization. The results suggest that for gamma sterilization, there were more carbonyl groups relative to anhydride groups in the unhydrolyzed gamma irradiated surface compared to the unhydrolyzed unirradiated surface, by comparing the relative intensities of 1726 to 1857 cm⁻¹.

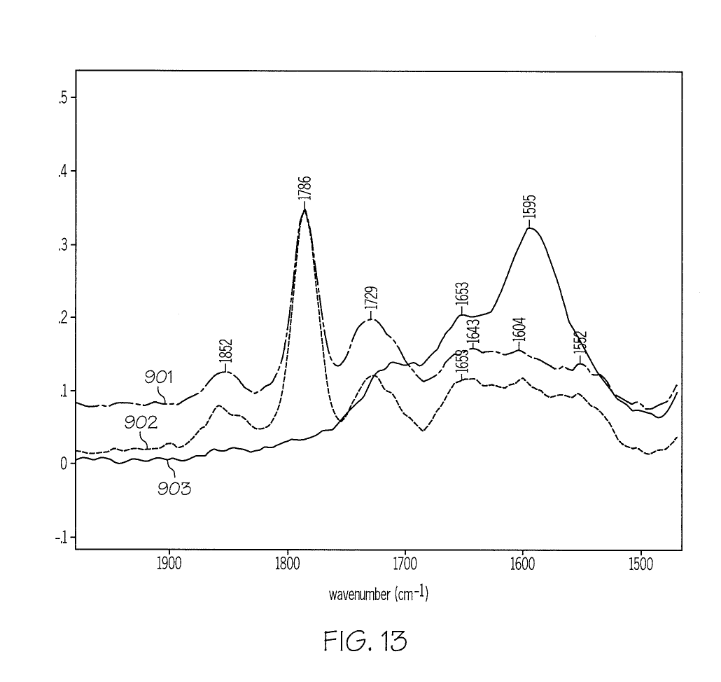

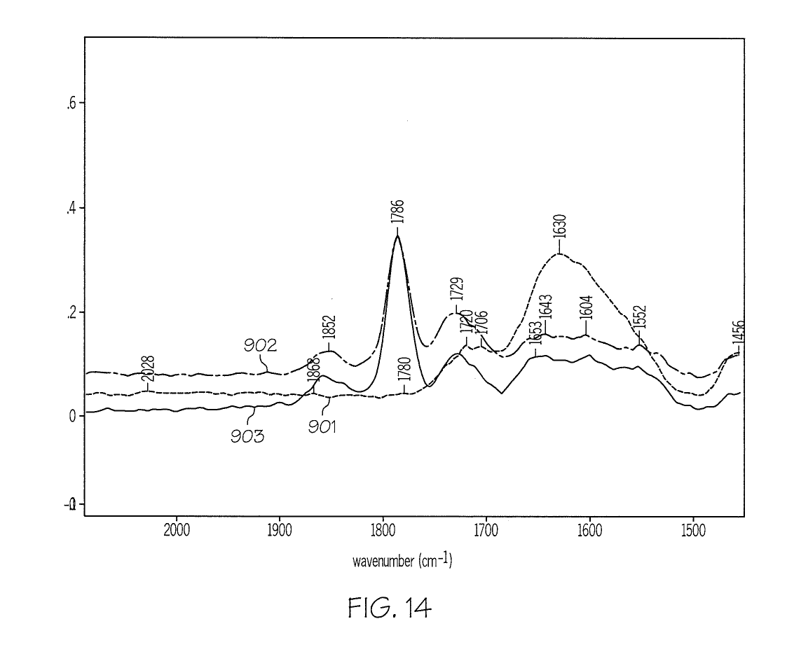

The effect of gamma irradiation alone and after hydrolysis with either phosphate buffer or borate buffer is shown in FIGS. 13 and 14, respectively. Both FIGS. 13 and 14 compare the unhydroyzed gamma irradiated surface with that which has been hydrolyzed and then gamma sterilized. As with the previous examples, the surface was polyethyelene alt maleic anhydride which has been derivatized about 40% with n-propyl amine. While gamma sterilization alone appears to generate more carbonyl groups compared to anhydride groups when comparing the relative intensities of 1729 to 1852 cm⁻¹, 901 and 902, respectively, hydrolysis eliminates the anhydride peak at 1852 and 1786 cm⁻¹ entirely, 903.

### Example 3

Analysis of cell culture on APS dEMA substrates. Cells cultured on the plates were either primary liver cells, or the liver cell line HepG2 C3a. For HepG2 C3a cells (ATCC Catalog #CRL-10741) culture took place in MEME (ATCC Catalog #30-2003) +10% fetal bovine serum (Gibco Cataolog #16000) +1% Penecillin Streptomycin (Gibco Catalog #15140) on Corning Tissue Culture Treated T75 Flasks. Cell passages less than 20 were used for cell culture. Trypsin (Gibco Catalog #25300) was utilized to detach cells for T75 TCT flasks and seed substrates for testing. Seeding densities were 25K/well for LDH attachment studies. For primary cell culture cryopreserved primary hepatocytes (XenoTech, LLC) were thawed and purified using the Percoll Isolatiion kit (Xenotech, LLC). The viable cells were plated in 10% FBS containing MFE medium (Corning) or serum free MFE medium or HBSS on various APS/dEMA surfaces along with BD's Collagen I and Matrigel™ surface as controls. The seeding density was 60K cells/well in a 96 well format. The cells were allowed to attach for 18-24 hours at 37° C. The medium was switched to serum free MFE medium at the second day for the rest of the culture period on the test surfaces.

Cell attachment was analyzed by an LDH Assay for cell attachment, conducted utilizing a kit from Promega's Cyto Tox 96 NonRadioactive Cytotoxicity Assay #G1780. FIG. 9 presents cell attachment data for cells grown in serum containing conditions as determined by the amount of LDH detected. As shown in FIG. 9, greater cell attachment for the unhydrolyzed APS dEMA surface compared to borate/water and to phosphate was observed for APS dEMA coated on Topas® suggesting an inverse relationship between carboxylic acid salt and HepG2 C3A cell attachment. The effect of adding galactose and leucine dimer together or leucine trimer to the substrates on cell attachment as measured by levels of LDH is shown in FIG. 10. Attachment was measured at day 1 (hatched bars) of culture on the different surfaces and retention at day 7 (open bars). The base surface chemistry attachment and retention was compared to that observed with Collagen I on the respective day and is reported as % Collagen I attachment/100.

The gene expression levels of primary human hepatocytes cultured on different substrates was assessed using quantitative real-time PCR method. Briefly, total RNA was first isolated from primary human hepatocytes using RNAquesous-96 kit (Applied Biosystems) and quantitated using Quanti-iT Ribogreen RNA Reagent Kit (Invitrogen). cDNA was then synthesized by TaqMan Reverse-Transcription Reagents. PCR reactions were prepared by adding cDNA to a reaction mixture containing the TaqMan PCR Master Mix solution and loaded in a custom-made TaqMan Low Density Array (microfluidic card). PCR amplified cDNAs were detected by real-time fluorescence on an Applied Biosystems 7900HT Fast Real-Time PCR System.

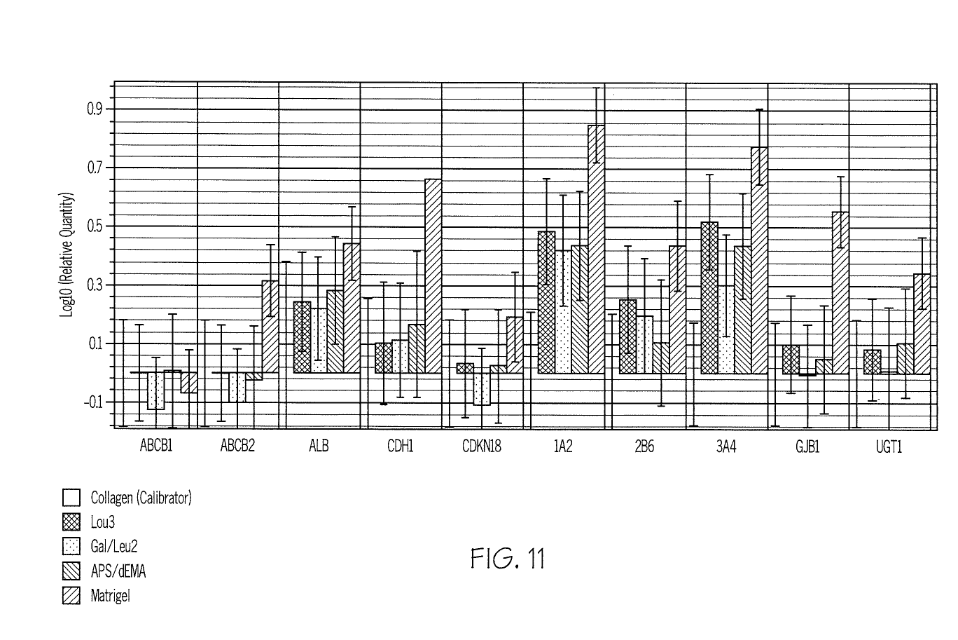

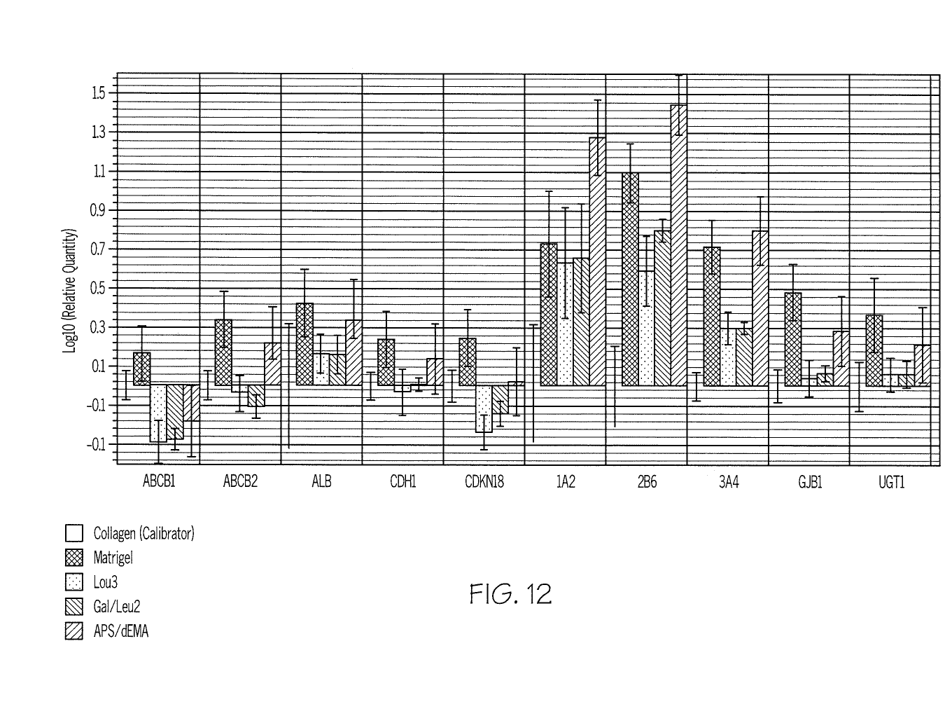

FIGS. 11 and 12 show the gene expression profile for primary liver cells cultured on various plates with (FIG. 11) or without (FIG. 12) serum. The figures show the gene expression profile for primary liver cells as cultured on Collagen I (shown with error bar at the Log10 intensity scale of zero, left), a leucine trimer derivative of APS/dEMA, a galactose/leucine dimer derivative of APS/dEMA, APS/dEMA and Matrigel™. Here ABCB1 and ABCB2 refer to liver cell transporters, ALB to albumin, CDH1 to E-Cadherin, CDKN18 to P27, 1A2, 2B6 and 3A4 to CYP1A2, CYP2B6 and CYP3A4, GJB1 to gap junction protein 1 and UGT1 to glucoronosyl transferase. CYP1A2, CYP2B6 and CYP3A4 are the cytochrome P450 enyzmes 1A2, 2B6 and 3A4. Primary liver cell gene expression for cells cultured on the substrates of the present invention exceeded that for cells cultured on Collagen I for CYP1A2, CYP2B6, CYP3A4, and ALB under full serum conditions. Primary liver cell gene expression for cells cultured on the substrates of the present invention was similar to cells cultured on Collagen I for ABCB1, ABCB2, CDH1, CDKN18, GJB1 and UGT1 under serum full conditions. Primary liver cell gene expression for cells cultured on the substrates of the present invention exceeded that for cells cultured on Matrigel™ for CYP1A2 and CYP2B6 under serum free conditions. Gene expression was similar for cells cultured on the substrates of the present invention to cells cultured on Matrigel™ for ABCB2, ALB, CDH1, CDKN18, CYP3A4, UGT1, GJB1 under serum free conditions.

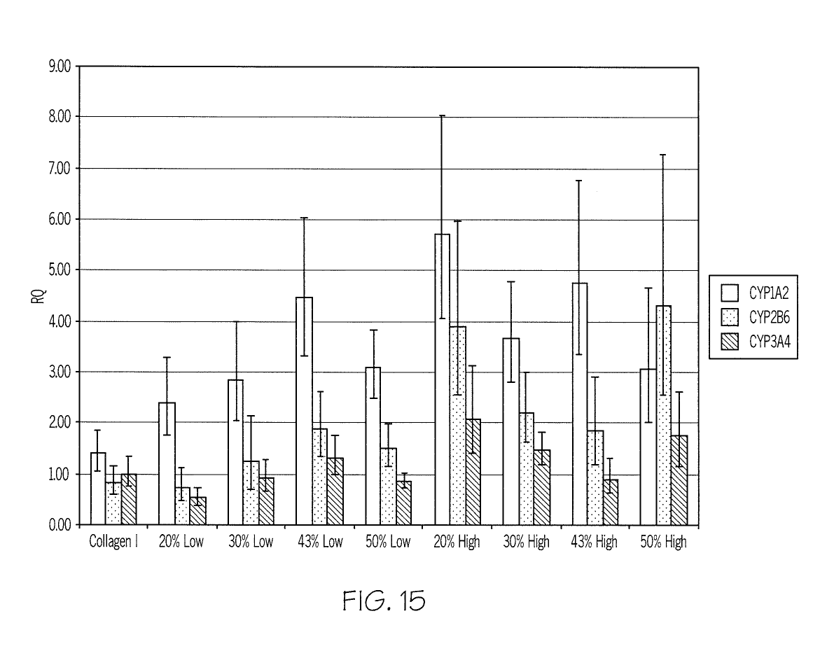

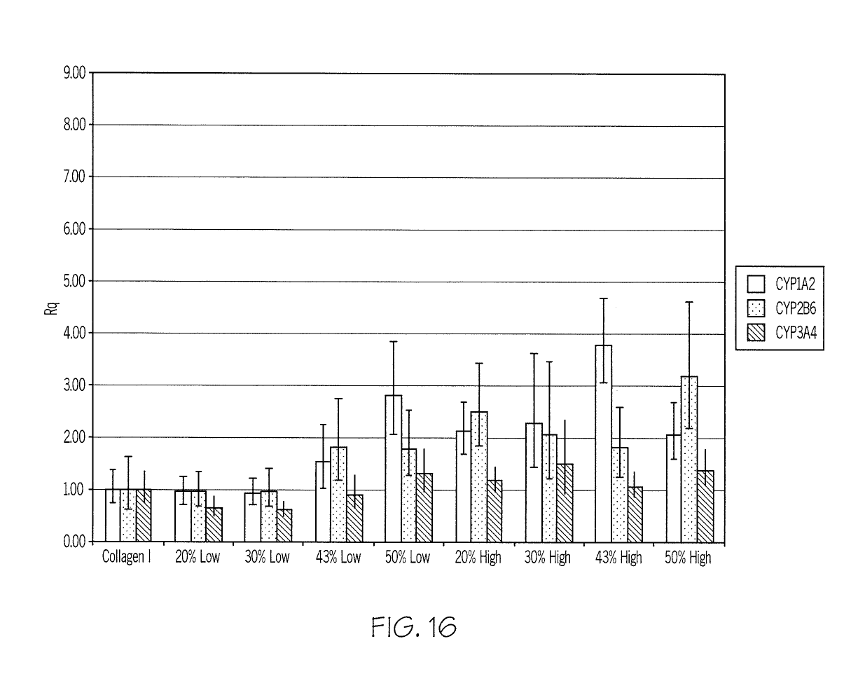

FIGS. 15 and 16 show the RTPCR response of three cytochrome P450 enzymes, CYP1A2, CYP2B6, and CYP3A4 with respect to Collagen I for gamma sterilized APS/dEMA under different derivatization conditions with n-propyl amine. The error bars represent 95% confidence level. The substrate was at either gamma high or gamma low sterilization levels under serum free conditions (FIG. 15) or serum full conditions (FIG. 16). Under serum free conditions and for gamma low sterilization (10-18 kGY), the best response is seen at 43% dEMA. For gamma high, (25-40 kGY) the functional response with respect to derivatization is less clear with the 20% derivatization level showing the highest response on average. Serum free conditions are, in general, better than serum full. Error bars represent a 95% confidence level in the data. For both cases, primary human liver cells (Xenotech, Lot #770) were cultured in MFE Medium (Corning Proprietary) under serum full or serum free conditions for 7 days prior to functional analysis. Serum full conditions represent a 10% fetal bovine serum level.

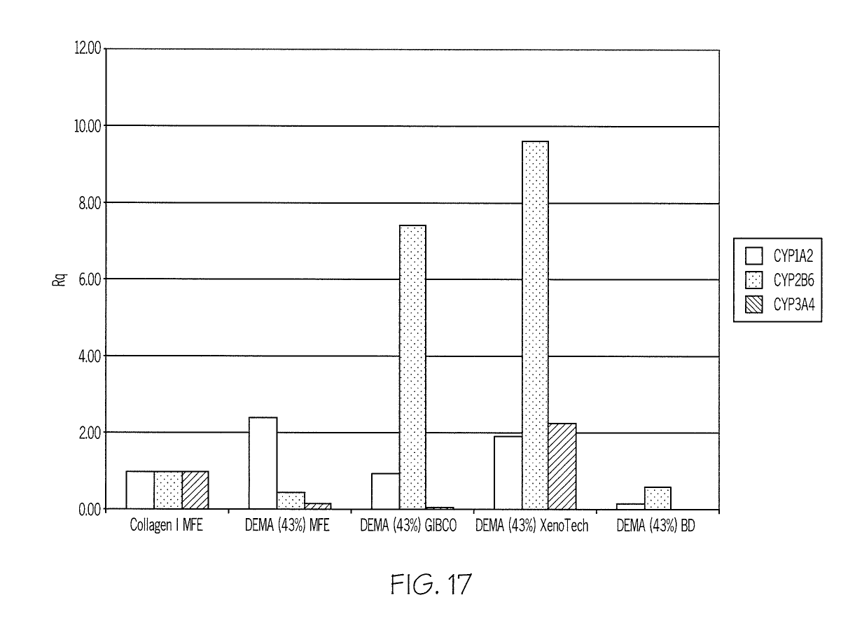

FIG. 17 shows RTPCR data for cells cultured under different media conditions using a APS dEMA substrate. 43% APS/dEMA was sterilized under low gamma conditions (10-18 kGY) and compared to Collagen I in MFE Medium for three cytochrome P450 enzymes, CYP1A2, CYP2B6, and CYP3A4. All of the 43% APS/dEMA was done under serum full conditions. Medium conditions were MFE (Corning Proprietary Media, Trademarked), Gibco™ Hepatozyme SFM (Catalog #17705), Xenotech Hepatocyte Culture Media (Catalog #K2300) or BD™ Hepatocyte Culture Media Kit (Catalog #355056). For this case, primary human liver cells (Xenotech, Lot #770) were cultured in the respective media for 7 days prior to functional analysis. Serum full conditions represent a 10% fetal bovine serum level.

FIG. 18 shows the basal gene expression of cytochrome P450 enzymes CYP1A2, CYP2B6 and CYP3A4 in primary cells cultured on surfaces having 0-90% derivatization, serum and serum free, using low gamma sterilization. FIG. 19 shows the basal gene expression of cytochrome P450 enzymes CYP1A2, CYP2B6 and CYP3A4 in primary cells cultured on surfaces having 0-90% derivatization, serum and serum free, using high gamma sterilization. Cell function improved as the percentage of derivatization (inactivation) increases. FIG. 18 shows that, particularly for CYP34A basal gene expression, basal gene expression improves between 43 and 90% derivatization or inactivation.

It will be apparent to those skilled in the art that various modifications and variations can be made to the present invention without departing from the spirit and scope of the invention. Thus it is intended that the present invention cover the modifications and variations of this invention provided they come within the scope of the appended claims and their equivalents.

What is claimed is:

1. A substrate for cell culture and cell-based assays comprising:
a support;
a tie layer comprising an an aminoalkylsilane or derivatives thereof attached to the support;
a synthetic polymer layer attached to the tie layer, the synthetic polymer layer comprising a plurality of ionizable hydrophilic groups, ionizable hydrophobic groups, or combinations;
wherein at least 50% of the ionizable hydrophilic groups, ionizable hydrophobic groups or combinations are inactivated.

2. The substrate of claim 1 wherein the support comprises a cell culture surface, a cell-based assay surface, a microplate, a slide, a strip well, a Petri dish, a flask, a multi-layer cell culture device, or a cell chamber in a fluidic device.

3. The substrate of claim 1 wherein the support comprises glass, plastic, polymeric resin, ceramic or combinations thereof, made in a flat, fibrous, or 3 dimensional format.

4. The substrate of claim 1 wherein the tie layer comprises aminopropylsilane or aminoalkylsilsesquioxane.

5. The substrate of claim 1 wherein the ionizable hydrophilic and ionizable hydrophobic groups comprise maleic acid, acrylic acid, n-acryloxysuccinamide, methacrylic acid, sulfonic acid or phosphonic acid, ethyelene, styrene, octadecene, methyl vinyl ether, isobutylene, vinyl ester, vinyl amide, acrylamide, acrylate groups, alkyl, allyl, aryl, or cycloalkyl groups, ethyl amine, propyl amine, butyl amine, pentyl or higher order amines.

6. The substrate of claim 1 wherein the synthetic polymer comprises maleic acid alt-copolymers, N-succinimide copolymers or derivatives thereof.

7. The substrate of claim 1 wherein the synthetic polymer layer comprises poly(ethylene-alt-maleic anhydride, poly(methyl vinyl ether-alt-maleic acid), poly(styrene-alt-maleic acid), maleic acid vinyl acetate copolymer, the n-alkyl amine and acid derivatives thereof or any mixture thereof.

8. The substrate of claim 1 wherein the synthetic polymer layer comprises poly(meth)acrylate-co-N-acryloxysuccinimide, polyacrylamide-co-N-acryloxysuccinimide, the n-alkyl amine or acid derivatives thereof or mixtures thereof.

9. The substrate of claim 1 further comprising small molecules attached to the synthetic polymer layer.

10. The substrate of claim 9 wherein the small molecules comprise peptides, proteins, biological ligands and sugar moieties.

11. The substrate of claim 9 wherein the small molecules comprise a YIGSR peptide, an RGD peptide, glucose, galactose, N-acetylgalactose, derivatives thereof or mimics thereof.

12. The substrate of claim 1 wherein the synthetic polymer layer has a surface contact angle of from about 10° to about 30°.

13. The substrate of claim 1 wherein the synthetic polymer layer is an n-propyl amine derivatized ethylene maleic acid layer.

14. A method for performing cell culture and cell-based assays comprising:
adhering at least one cell to a substrate in the absence of serum proteins, wherein the substrate comprises a support, a tie layer comprising an aminoalkylsilane or derivatives thereof attached to the support, a synthetic polymer layer attached to the tie layer, the synthetic polymer surface comprising a plurality of ionizable hydrophilic groups, ionizable hydrophobic groups or combinations, wherein at least 50% of the ionizable hydrophilic groups, ionizable hydrophobic groups, or combinations are inactivated;
culturing the cell on the substrate without serum proteins; and
performing a cell-based assay.

15. The method of claim 14 wherein the cell is a mammalian cell.

16. The method of claim 14 wherein the synthetic polymer layer comprises maleic acid alt-copolymers or N-succinimide copolymers.

17. The method of claim 14 wherein the substrate further comprises small molecules attached to the synthetic polymer layer, the small molecules comprising peptides, proteins, sugars or combinations thereof.

18. A method of producing a substrate for cell culture and cell-based assays comprising:
attaching a tie layer to a support, wherein the tie layer comprises an aminoalkylsilsesquioxane or derivatives thereof;
attaching a synthetic polymer layer to the tie layer, the synthetic polymer surface comprising a plurality of ionizable hydrophilic groups, ionizable hydrophobic groups or a combination; and
irradiating the substrate to inactivate at least 50% of the ionizable hydrophilic groups, ionizable hydrophobic groups or combinations.

19. The method of claim 18 wherein the synthetic polymer layer comprises maleic acid alt-copolymers or N-succinimide copolymers.

20. The method of claim 18 wherein the treating step is hydrolysis, UV treatment or gamma irradiation.
